(19) United States
(12) Patent Application Publication
(10) Pub. No.: US 2011/0305764 A1
(43) Pub. Date: Dec. 15, 2011

(54) Use of a Mare's Milk Concentrate Dried on a Highly-Dispersed, Biologically Inert Matrix

(75) Inventors: Bodo Kuklinski, Rostock (DE); Raimund Schiefer, Tamsweg (AT); Gertrude Markolin, Mariapfarr (AT); Peter Kössler, Mariapfarr (AT); Norbert Fuchs, Mariapfarr (AT)

(73) Assignee: Nutropia Ernahrungsmedizinische Forschungs GmbH, Unternberg (AT)

(21) Appl. No.: 13/215,960

(22) Filed: Aug. 23, 2011

Related U.S. Application Data
(63) Continuation of application No. 10/511,882, filed on Oct. 19, 2004, now abandoned.

(30) Foreign Application Priority Data
Apr. 25, 2002 (AT) A 640/2002

(51) Int. Cl. A61K 35/20; A61P 17/06; A61P 17/00; A61K 9/14

(52) U.S. Cl. 424/490; 424/535

(57) ABSTRACT

The invention relates to the use of a mare's milk concentrate dried on a highly-dispersed, biologically inert matrix, for producing a preparation for the treatment of skin diseases, especially dry skin diseases.

The present invention relates to the use of a mare milk concentrate dried on a biologically inert, highly disperse matrix.

Neurodermatitis (syn. atopic dermatitis; atopic eczema; endogenous eczema) is a chronic or chronically recurring skin disease. In the early infancy neurodermatitis becomes clinically apparent by itching, redness, scaling, exudation and incrustation primarily on the cheeks (milky tetter), on the ears or in various fold regions. Those mild forms of neurodermatitis are frequently not diagnosed as neurodermatitis and consequently not adequately treated. Beginning with approximately the second year of age, the clinical picture of neurodermatitis corresponds to that of adults with flexion eczemas (Exzema flexurarum) being predominant at that stage. Later on, at school age and during puberty, a third form of progress appears as “Neurodermatitis disseminata”, whereby the whole body may be afflicted with eczematic focuses (face, trunk, extremities, articular bends).

Its etiopathogenesis is considered as largely unidentified with the following factors being under discussion as potential causes and/or promoters of the clinical characteristics of the disease: genetic predisposition (autosomally dominant inheritance), neurovegetative regulatory disturbances of the vasomotor functions, psychic factors (professional and/or family-related changes, overload, problems with the partner or family), exogenous factors (allergenes, climate), intestinal candidosis, immunological factors (immediate-type IGE-mediated hypersensitivity reactions, or Type I allergies) as well as enzymatic defects (limited activity of the enzyme delta-6-desaturase).

In line with the multifactorial genesis of neurodermatitis, the therapeutic offer is accordingly manifold: symptomatic (internal and external) treatment with antihistamines, (internal and external) glucocorticoids, benzodiazepines (the agonizing pruritus occurring mainly during the night), oil and tar baths, climatotherapies in mountainous and maritime climates, urea-containing substances for external application, (internal and external) antimycotic agents, UV therapy as well as linolenic acid-containing vegetable oils for internal use.

All over Europe, six to eight million patients are said to suffer from neurodermatitis, some three hundred thousand new diseases being reported per year. While only 0.7% of the affected population are adults, the portion of European children suffering from atopic diseases ranges between 10 and 15% such that neurodermatitis constitutes primarily a pediatric problem.

On the physiologic and biochemical levels, the limited activity of the enzyme delta-6-desaturase is being discussed as the possible source of trouble. This enzyme catalyzes the transformation of the essential omega-6 fatty acid “linolic acid” (C18:2) into gammalinolenic acid (C18:3), which is, in turn, elongated to dihomo-gamma-linolenic acid (C20:3) in a subsequent step, constituting the physiologic starting product for series one prostaglandins (PGE₁). Series one prostaglandins exhibit inflammation-inhibiting and vasodilative activities and are reduced in atopic patients as against healthy people. Since atopic patients exhibit increased concentrations of linolic acid on the one hand and gamma-linolenic acid levels reduced by more than a half in the plasma on the other hand, the “delta-6-desaturase hypothesis” is deemed largely assured. The catalytic functions as well as the activities of delta-6-desaturases are dependent on iron (hemin- and non-hemin-bound), niacin (NADH or NADPH) as well as riboflavin (FADH₂). From the above-mentioned dihomo-gamma-linolenic acid, not only PGE but also arachidonic acid (C20:4) is formed, which, in turn, is the biochemical precursor of prostacyclins, thromboxanes and leucotrienes. The extent by which the pathologic events of neurodermatitis are influenced by immunomodulators is still under investigation.

According to other studies, neurodermatitis is supposed to be based on a pathophysiologic maturation disorder of the T lymphocytes contained in the thymus and/or epidermis.

This maturation disorder causes uncontrolled cutaneous T-cell infiltration. In any event, it is taken for sure that the essential fatty acids (the omega-3 fatty acid “alpha-linolenic acid” as well as the omega-6 fatty acid “linolic acid”) and the eicosanoids formed thereof substantially influence the integrity of the epidermis and the efficiency of the immune system alike. In doing so, the immunoregulatory effects, particularly those of the essential omega-6 fatty acids, are mediated and modulated by series one prostaglandins (PGE₁).

Another additional complication involved in neurodermatitis is the occurrence of bacterial or viral secondary infections provoked by constant scratching on the affected, itching skin sites.

Psoriasis (psora) is one of the most frequently occurring skin diseases of adults. One to two percent of all Europeans are afflicted with this intermittently occurring skin disease, which is not transmissible. It is likely to be caused by an immunopathogenetic event occurring in the skin and leading to an inflammation and massive hyperproliferation of keratinocytes, and hence a superfast formation of the epidermis. Presumably, this is due to genetic factors.

Inflammatory processes, lesions and psychosomatic disturbances promote the outbreak of the disease. The therapy of psoriasis is determined by two essential factors. For one part, it is a chronically recurring disease which may call for treatment over a very long period of time, for the other part individual factors like internal concomitant affections as well as clinical forms of psoriasis and pretreatment have to be taken into account. Therapeutic forms comprise local therapy and/or systemic therapy as well as phototherapy, which may be combined with other therapies. They will bring about alleviation, but no healing of the disease.

Its phenotypical expressivity and course are variable. Light forms of progress show individual focuses on predilection sites, which can persist for years or alternate with differently long periods of complete freedom of symptoms. Severe forms are characterized by extensive psoriatic efflorescences, the spontaneous regression of lesions being rare. The severest expressivity comprises erythrodermia as well as generalized pustulous psoriasis. Both forms show general signs. In terms of clinical picture, Psoriasis vulgaris is the most frequent form of psoriasis, occurring in 90%. The typical morphology is characterized by sharply limited erythematous papules and plaques involving coarsely lamellar, silver-shining exfoliation. The predilection sites are the extension sites of the elbows and knees, both periumbilical and sacral, yet extended focuses are also frequently found on the scalp. Psoriasis guttata (eruptive, small-spot psoriasis) develops primarily in younger patients following streptococcus infections of the upper airways, as a primary manifestation. The generalized Psoriasis pustulosa (by Zumbusch) is the severest form of psoriasis, with the total integument being intermittently transformed into pustules with concomitant fever attacks. Localized forms comprise Pustulosis palmoplantaris on the palms and soles and the very rare Acrodermatitis continua suppurativa. 10 to 30% of the patients suffering from psoriasis are also affected by psoriasis arthritis. In most cases, this goes hand in hand with psoriatic changes of the finger and toe nails and may precede skin changes.

Dietetics reports have described the successful application of native mare milk also in the case of neurodermatitis as well as psoriasis amongst others. Unlike cow milk, mare milk has a composition very similar to that of human milk, including a higher portion of essential, highly unsaturated fatty acids as well as phospholipids, which are necessary for the metabolism of the skin, although the absolute fat content of mare milk is lower than that of cow milk. In addition, mare milk also contains higher-than-average portions of natural antioxidative nutrients like E vitamins, vitamin C and vitamin B12.

In “Zur Verwendbarkeit von Stutenmilch, Kumyβ und Eselmilch als Diätetika und Heilmittel unter besonderer Berücksichtigung der Bedürfnisse des Säuglings und des Frühgeborenen” (Verlag Dr. Markus Hänsel-Hohenhausen (1996), pages 367-376), Alexander Bühlbäcker describes, for instance, the use of native mare milk as a food additive in the treatment of neurodermatitis. From those case descriptions it is apparent that, in the dietetic treatment of neurodermatitis with native mare milk, a minimum treatment time of ten months is required and mare milk is ineffective if given alone, i.e., without additional therapeutic and dietetic measures. Furthermore, native mare milk involves a storage problem, being not storage-stable at room temperature. Native mare milk is stable for only a few days at room temperature, about one week in the cooled state, and a maximum of half a year in the deepfrozen state.

In order to circumvent the problem of low storage stability, dried mare milk products and, in particular, powders or capsules have been produced. Drying in those cases is effected, for instance, by freeze-drying, which is, however, uneconomical, by spray-drying, which entails the destruction of high-grade proteins, and by evaporation, which leaves an amorphous mass offering limited storability.

Therefore, there has been the need for a preparation being an alternative to native mare milk for the treatment of (dry) skin diseases and, in particular, neurodermatitis and psoriasis, which is to be effective without any additional therapeutic and dietetic measures, whereby healing or improvement is to commence already after a short treatment period and the product to be administered is to be resistant to storage over an extended term even at room temperature. Furthermore, it is important that such a stable product has a high biologic value.

This object is achieved by the use of a mare milk concentrate dried on a biologically inert, highly disperse matrix for the production of a preparation for the treatment of skin diseases and, in particular, dry skin diseases.

By “dry skin diseases”, dry aged skin, psoriasis, neurodermatitis and the like are, for instance, understood.

It has been shown in a surprising manner that a (stable) mare milk concentrate dried on a biologically inert, highly disperse matrix is perfectly suitable for the treatment of skin diseases and, unlike native mare milk, offers a high storage stability even at room temperature.

The mare milk dry concentrate obtained by the method according to the invention has a stability of 24 to 36 months. This technological procedure, therefore, enables mare milk to be combined with other functional nutrients (in the specific case with skin-effective vitamins, minerals, trace elements, highly unsaturated fatty acids).

As pointed out above, the production of such mare milk concentrates is already known, since technological methods have been developed to extend the stability of mare milk at room temperature from a few days to at least two years without destroying the temperature- and oxygen-sensitive ingredients of mare milk during the drying process.

To this end, vacuum evaporation methods have, for instance, been already described to remove the water contained in the milk at a temperature of below 40° C. and under the exclusion of oxygen, thus drying and concentrating the mare milk. Due to its content of low-molecular oligosaccharides, oligopeptides as well as high-quality oils, the mare milk concentrate is present as a viscous amorphous mass which is only difficult to galenically process in this form. In order to compensate for this technological drawback, it is taught, for instance, in AT 393 961 to supplement mare milk with inert, highly disperse silicon dioxide (silica) as a matrix before subjecting it to vacuum distillation, so that a crystalline, powdery dry concentrate will be obtained after vacuum distillation.

These mare milk dry concentrates based on highly disperse matrices have, thus, been developed in order to simplify the preparation process while preserving the high-quality ingredients and also to enable mare milk to be stored over extended periods of time without any quality losses. The silicon dioxide, furthermore, imparts an enhanced flowability on the product. This dry milk concentrate is described to be used as an immune stimulator. The use of this special concentrate for the treatment of skin diseases has, however, so far been neither described nor rendered obvious.

It has now turned out for the first time in a surprising manner that this special mare milk dry concentrate is particularly well apt for the treatment of skin diseases. Compared to, for instance, freezedried mare milk, the mare milk concentrate according to the invention offers advantages in the treatment of skin diseases, since the biologic value of native mare milk is fully retained by the careful drying rendered feasible on account of the uniform distribution of the mare milk on the highly disperse matrix.

The term “highly disperse matrix” according to the invention serves to denote a matrix having a large surface area of at least 50 m²/g. In this context, it is important that the matrix is biologically inert such that the mare milk will not be chemically altered and hence loose some of its biologic value. By drying the mare milk on a highly disperse matrix, it is ensured that the mare milk droplets will accumulate on the matrix particles in a finely distributed manner and hence provide the optimum fine surface distribution of the milk required for careful drying. The milk is, thus, distributed in a manner as intensely as possible on a volume as small as possible. This enables the milk to be dried rapidly under gentle conditions and made available in a high concentration and a storage-stable form. The matrix not only causes the milk to be finely distributed on as large a surface as possible, but also offers a certain protection against other substances attacking the sensitive ingredients of the milk like, for instance, the unsaturated fatty acids. The milk can be applied on the highly disperse matrix, for instance, by spraying.

By drying the mare milk in this manner, it is feasible to concentrate and dry its temperature- and oxygen-sensitive ingredients, particularly its fatty acids, carefully without any loss such that the high-quality ingredients are dried while applying mild temperatures. From this results a mare milk concentrate which not only offers a maximum biologic value, but is also storage-stable at room temperature and surprisingly better suited for the treatment of skin diseases than conventional preparations. Compared, for instance, to a treatment with spray-dried mare milk, the use according to the invention entails rapid improvement of the disease and also healing for an extended period of time.

Furthermore, another advantage of the concentrate according to the invention resides in the option to combine this valuable natural product with biologically active additives as desired, to thereby develop and distribute biologically active and marketable products.

By taking a biologically high-grade concentrate, very large amounts of biologically active ingredients corresponding to very large amounts of native mare milk can be supplied every day. Consequently, the treatment becomes simpler and more pleasant for the patient.

The preparation may, for instance, be provided in the form of a powder, tablet or capsule and further processed directly before its use, for instance, with water to form a cream or milk. The preparation is above all intended for oral intake. It goes without saying that the mare milk concentrate in the form of a cream or lotion may also be applied on the skin sites to be treated.

The average particle size of the matrix is, for instance, about 900 nm at most, preferably about 500 nm at most, in a particularly preferred manner 250 nm at most, 100 nm at most, 50 nm at most, 25 nm at most and, in the most preferred manner, 15 nm at most.

Preferably, the matrix has an average surface area of at least 100 m²/g and, in a particularly preferred manner, at least 150 m²/g and, in an even more preferred manner, at least 200 m²/g and, in the most preferred manner, at least 400 m²/g.

The mare milk can, for instance, be applied on the matrix via jet nozzles, and this mixture can then be carefully dried in a mixing vessel, e.g. a mixing screw, for instance by applying vacuum drying. The vapor formed by vacuum drying may, for instance, be condensed in a condenser and carried off into a water reservoir.

The drying vessel is preferably arranged in a rotational and horizontal manner and may be of any dimension such as, e.g., about 500 to 1000 L. The unit is preferably controlled in terms of temperature and pressure. Furthermore, it is beneficial if additional parameters such as the mixing time, injection time, injection pressure, tilting angle, vibrators, shearing head activation, etc. are programmable and regulatable. This helps to optimize the method, the optimum values being readily adjustable by the person skilled in the art.

In a particularly preferred manner, the preparation is used for the treatment of neurodermatitis or psoriasis. These skin diseases belong to what is called “dry skin diseases”. It has been shown that the mare milk concentrate dried on a biologically inert, highly disperse matrix is particularly apt for the treatment of neurodermatitis and psoriasis. As already described above, it is known from the prior art to use native mare milk for the treatment of neurodermatitis and psoriasis. Yet, it could be proved that the carefully dried mare milk concentrate according to the present application is particularly suitable, because, it induces quicker healing or improvement of the illness than, for instance, spray-dried mare milk, and also does not require any additional therapeutic or dietetic measures as against treatments with mare milk dried in any other manner. Unlike native mare milk, the carefully dried concentrate is stable and comprises biologically high-grade ingredients in a highly concentrated form.

A particularly beneficial use is provided in that the matrix is comprised of highly disperse silicon dioxide. This matrix is biologically inert and highly disperse to a sufficient extent so as to be perfectly suited for the careful drying of mare milk. Moreover, silicon dioxide is useful for the production of a preparation to be taken orally, because silicon dioxide is completely safe from a medical point of view.

The matrix is, for instance, made of Aerosil®, a highly disperse silica with a content of SiO₂ of more than 99.8%. This matrix is composed of amorphous spherical particles having diameters of about 10 to 20 nm. At a volume of about 15 ml, 1 g Aerosil® has a surface area of 100 to 400 m². This matrix is particularly suitable for the use according to the invention.

A particularly advantageous use is, moreover, characterized in that the mare milk concentrate was dried at a temperature of from 10 to 50° C. and, in particular 35 to 40° C. This temperature range safeguards completely careful drying so as to preserve the biologic value of the mare milk. At these temperatures, all of the important and also sensitive ingredients will be preserved. In this respect, the mixing vessel containing the highly disperse matrix and the mare milk can be heated to a constant temperature, for instance, by means of control.

It is, furthermore, beneficial if the mare milk concentrate was dried at a pressure of from 1 to 50 mbar and, in particular 10 to 30 mbar. Within this pressure range, the biologically relevant ingredients and, in particular, the unsaturated fatty acids will be preserved undamaged. Furthermore, this pressure range safeguards careful drying without temperature impairment.

In a preferred manner, the preparation additionally comprises essential fatty acids and, in particular, vegetable essential fatty acids. In particular, these include linolenic acid, stearidonic acid, eicosadienoic acid, linolic acid, palmitoleic acid, vaccenic acid, eicosenic acid, erucic acid, nervonic acid, oleic acid. The combination of a dried mare milk concentrate with vegetable essential fatty acids has turned out to be of particular benefit to the treatment of skin diseases, because thereby all of the substances necessary for the healing of such a disease will be administered at a time. The vegetable essential fatty acids supplement the mare milk concentrate in the optimum manner.

It is, furthermore, advantageous if the preparation additionally contains at least one substance selected from the group consisting of hydrogen carbonate, potassium, carbonate, citrate, calcium, magnesium, vitamin C, vitamin E, niacin, zinc, iron, beta-carotene, pantothenic acid, manganese, vitamin B6, vitamin B2, vitamin B1, copper, sodium, biotin, folic acid, molybdenum, selenium, xanthan, fructose, citric acid and vitamin B12 or a combination of at least two of these substances.

If at least one substance, or a combination of at least two substances, of this group is added to the mare milk concentrate, an extremely efficient combination is thus made available, since the mare milk concentrate is supplemented in the optimum manner. Thus, a preparation is provided, which is excellently suitable for the treatment of skin diseases and, in particular, neurodermatitis and psoriasis.

The present invention will now be explained in more detail by way of the following examples, to which, however, it shall not be limited.

### EXAMPLES

### Treatment of Psoriasis and Neurodermatitis Patients with a Mare Milk Concentrate

Patients suffering from psoriasis and neurodermatitis were treated with a mare milk concentrate (“neurodermatitis cocktail”), this concentrate comprising the ingredients indicated in Table 1. This concentrate was carefully prepared by applying native mare milk on a highly disperse silicon dioxide matrix in a finely distributed manner and gently drying the same in a mixing vessel at about 32° C. and 10 mbar. 150 kg of mare milk were pasteurized and subsequently supplemented with 625 g of highly disperse silicon dioxide (as the inert carrier matrix) as well as 0.75 g of citric acid and 7.50 g of D,L-alpha-tocopherol (as stabilizers). This mixture was concentrated to dryness in a closed evaporation unit at about 32° C. and a vacuum of 10 mbar under constant stirring for a period of 24 hours. After drying, the mare milk dry concentrate was mixed to a powder with the oils, minerals, vitamins and trace elements indicated in Table 1 as well as with highly disperse silicon dioxide as an auxiliary agent.

Recommended regimen: once a day, preferably at night before going to bed; stir one portion into water or milk by the aid of a shaker or stirring rod and drink in sips.

Children from age 1 to under 4: Stir 1 level tablespoon (about 6.67 g) powder into ⅛ L (125 ml) water or milk.

Children from age 4 to under 13: Stir 2 level tablespoons (about 13.3 g) powder into ¼ L (250 ml) water or milk.

Children from age 13, adolescents and adults: Stir 3 level tablespoons (about 20 g) powder into ¼ l (250 ml) water or milk.

TABLE 1
Nutrients per 1, 2, 3 level tablespoons of neurodermatitis cocktail
(tbsp. = tablespoon; corresponding to 6.67 g, 13.3 g and 20 g, respectively).

| | Children from | | Children from | | Children from age 13, | |
|---|---|---|---|---|---|---|
| | age 1 to under 4 | | age 4 to under 13 | | adolescents and adults | |
| Dosage□ | 1 tbsp. containing: | | 2 tbsps. containing: | | 3 tbsps. containing: | |
| Mare milk dry concentrate | 330 | mg | 660 | mg | 990 | mg |
| omega 3 FS □- | 280.1 | mg | 560.2 | mg | 840.3 | mg |
| Linolenic acid | | | | | | |
| omega 3 FS Stearidonic | 0.3 | mg | 0.5 | mg | 0.8 | mg |
| acid | | | | | | |
| omega 6 FS Eicosadienoic | 0.5 | mg | 1.1 | mg | 1.6 | mg |
| acid | | | | | | |
| omega 6 FS | 66.7 | mg | 133.3 | mg | 200.0 | mg |
| □□Linolenic acid | | | | | | |
| omega 6 FS Linolic | 448.2 | mg | 896.4 | mg | 1344.5 | mg |
| acid | | | | | | |
| Omega 7 FS Palmitoleic | 0.5 | mg | 1.1 | mg | 1.6 | mg |
| acid | | | | | | |
| Omega 7 FS Vaccenic | 1.3 | mg | 2.7 | mg | 4.0 | mg |
| acid | | | | | | |
| Omega 9 FS Eicosenic | 10.7 | mg | 21.3 | mg | 32.0 | mg |
| acid | | | | | | |
| Omega 9 FS Erucic | 8.0 | mg | 16.0 | mg | 24.0 | mg |
| acid | | | | | | |
| Omega 9 FS Nervonic | 5.3 | mg | 10.7 | mg | 16.0 | mg |
| acid | | | | | | |
| Omega 9 FS Oleic acid | 208.0 | mg | 416.0 | mg | 624.0 | mg |
| Total of essential | 1029.6 | mg | 2059.3 | mg | 3088.9 | mg |
| fatty acids | | | | | | |
| Hydrogen carbonate | 195.1 | mg | 390.3 | mg | 585.4 | mg |
| Potassium | 166.7 | mg | 333.3 | mg | 500.0 | mg |
| Carbonate | 88.7 | mg | 177.3 | mg | 266.0 | mg |
| Citrate | 67.1 | mg | 134.2 | mg | 201.3 | mg |
| Calcium | 66.7 | mg | 133.3 | mg | 200.0 | mg |
| Magnesium | 66.7 | mg | 133.3 | mg | 200.0 | mg |
| Vitamin C | 20.0 | mg | 40.0 | mg | 60.0 | mg |
| Vitamin E | 6.7 | mg | 13.3 | mg | 20.0 | mg |
| Niacin | 5.0 | mg | 10.0 | mg | 15.0 | mg |
| Zinc | 4.0 | mg | 8.0 | mg | 12.0 | mg |
| Iron | 3.3 | mg | 6.7 | mg | 10.0 | mg |
| Beta-Carotene | 2.0 | mg | 4.0 | mg | 6.0 | mg |
| Pantothenic acid | 1.7 | mg | 3.3 | mg | 5.0 | mg |
| Manganese | 0.67 | mg | 1.3 | mg | 2.0 | mg |
| Vitamin B6 | 0.53 | mg | 1.1 | mg | 1.6 | mg |
| Vitamin B2 | 0.50 | mg | 1.0 | mg | 1.5 | mg |
| Vitamin B1 | 0.37 | mg | 0.73 | mg | 1.1 | mg |
| Copper | 0.17 | mg | 0.33 | mg | 0.5 | mg |
| Sodium | 35 | mcg | 71 | mcg | 106 | mcg |
| Biotin | 33 | mcg | 67 | mcg | 100 | mcg |
| Folic acid | 33 | mcg | 67 | mcg | 100 | mcg |
| Molybdenum | 33 | mcg | 67 | mcg | 100 | mcg |
| Selenium | 33 | mcg | 67 | mcg | 100 | mcg |
| Vitamin B12 | 0.7 | mcg | 1.3 | mcg | 2 | mcg |

Investigation parameters:

Neurodermatitis

Main target parameters: SCORAD (Severity Scoring of Atopic Dermatitis). The SCORAD index (Severity Scoring of Atopic Dermatitis) was used to qualitatively and quantitatively assess the degree of severity of the atopic eczema. It allows the standardized judgment of the degree of intensity of six typical morphologic changes (0-3, max. 18), the portion of the affected skin area (%) and the subjective assessment of itching and sleep loss using a visual analog scale (0-10, max. 20). Analyses of individual, as well as contextual groups of, parameters or the total score (maximum: 103 scores) are feasible.

The SCORAD is based on information as to the extension (A), intensity (B) and symptoms (C) such as pruritus and insomnia. As is readily apparent from the SCORAD formula A/5 plus 7B/2 plus C, intensity is attributed the strongest weighting. Five different main signs (erythema, edema/papule formation, exudation/-incrustation, excoriation and lichenification) are represented for each degree of severity. The patients have to enter their symptoms on a visual analog scale themselves.

Secondary parameters: Compatibility and acceptance of the test substance.

SCORAD calculations were done by means of the SCORAD calculator of the University of Nantes (http://scorad.sante.univ-nantes.fr/Compute.html).

Psoriasis:

Main target parameters: The degree of spreading and the intensity of typical morphologic changes of the afflicted skin surface were assessed. The Psoriasis Area and Severity Index (PASI) served as a measuring instrument. This index takes into account the surface area of the affected skin as well as the extent of inflammation and excessive cell division. To this end, the investigator determines redness, thickening and scaling for one focus each on the head, trunk, arm and leg, using a scale from 0 to 4. The counts are multiplied by those of the estimated affection. From this results a PASI of between 0 and 96 for the percentage portion of each individual region according to a conversion formula.

Secondary parameters: Compatibility and acceptance of the test substance.

### Example 1

Participant No. 01

Initials: JT

Date of birth: Sep. 14, 1991

Sex: male

Diagnosis: neurodermatitis since birth

Dosage: neurodermatitis cocktail: 2 tablespoons per day (=13.3 g)

TABLE 2
SCORAD - Patient No. 01

| | | 1^{st} visit | 2^{nd} visit | 3^{rd} visit |
|---|---|---|---|---|
| | Baseline | after 1 | after 2 | after 3 |
| | visit | month | months | months |
| A: Extent (0-102) | 6 | 0 | 0 | 0 |
| B: Intensity | 7 | 2 | 2 | 2 |
| (0-18) | | | | |
| C: Personal symptoms | 5 | 2 | 0 | 0 |
| (0-20) | | | | |
| SCORAD* (0-103) | 31 | 9 | 7 | 7 |

*SCORAD = A/5 + 7B/2 + C

Extent, intensity, personal symptoms and total score showed marked improvements in the therapy progress. Accompanying measures comprised the patient's application of greasing ointments during the supplementation period.

In the course of the study, the patient did not report any side effects of the preparation. In terms of taste, the preparation was rated “good” by the patient.

### Example 2

Participant No. 02

Initials: RA

Date of birth: Apr. 18, 1998

Sex: female

Diagnosis: neurodermatitis since birth

Dosage: neurodermatitis cocktail: 1 tablespoon per day (=6.67 g)

TABLE 3
SCORAD - Patient No. 02

| | | 1^{st} visit | 2^{nd} visit | 3^{rd} visit |
|---|---|---|---|---|
| | Baseline | after 1 | after 2 | after 3 |
| | visit | month | months | months |
| A: Extent | 8 | 3 | 1 | 1 |
| (0-102) | | | | |
| B: Intensity | 6 | 2 | 2 | 2 |
| (0-18) | | | | |
| C: Personal symptoms | 7 | 0 | 0 | 0 |
| (0-20) | | | | |
| SCORAD* (0-103) | 30 | 8 | 7 | 7 |

*SCORAD = A/5 + 7B/2 + C

Extent, intensity, personal symptoms and total score showed marked improvements in the therapy progress.

Accompanying measures comprised the patient's application of greasing ointments and oil baths during the supplementation period.

In the course of the study, the patient did not report any side effects of the preparation. In terms of taste, the preparation was rated “very good” by the patient.

### Example 3

Participant No. 04

Initials: ZM

Date of birth: Feb. 17, 1968

Sex: female

Diagnosis: neurodermatitis since birth

Dosage: neurodermatitis cocktail: 3 tablespoons per day (=20 g)

TABLE 4
SCORAD - Patient No. 04

| | | 1^{st} visit | 2^{nd} visit | 3^{rd} visit |
|---|---|---|---|---|
| | Baseline | after 1 | after 2 | after 3 |
| | visit | month | months | months |
| A: Extent (0-102) | 12 | 6 | — | 1 |
| B: Intensity | 7 | 3 | — | 2 |
| (0-18) | | | | |
| C: Personal symptoms | 8 | 3 | — | 0 |
| (0-20) | | | | |
| SCORAD* (0-103) | 35 | 15 | — | 7 |

*SCORAD = A/5 + 7B/2 + C

Extent, intensity, personal symptoms and total score showed marked improvements in the therapy progress. Accompanying measures comprised the patient's application of greasing ointments and oil baths during the supplementation period.

In the course of the study, the patient did not report any side effects of the preparation. In terms of taste, the preparation was rated “very good” by the patient.

The three reported neurodermatitis cases initially showed moderately severe forms (SCORAD 30 to 35) of atopic dermatitis. The results of the neurodermatitis cocktail therapy in all of the participants in the study showed marked and sustained improvements in the skin findings which did not change till the end of the study (SCORAD 7—light form of neurodermatitis—after 12 weeks of supplementation).

Psoriasis

The PASI (Psoriasis Area Severity Index) was used to qualitatively and quantitatively assess the degree of severity of psoriasis.

PASI for the individual skin segments

(http://members.aol.com/psorsite/docs/pasi.html): Skin segment legs:

(itching_{legs}'redness_{legs}+scaling_{legs}+skin thickness_{legs})×spreading_{legs}×0.4=total_{legs}

Skin segment trunk:

(itchingₜᵣᵤₙₖ+rednessₜᵣᵤₙₖ+scalingₜᵣᵤₙₖ+skin thicknessₜᵣᵤₙₖ)×spreadingₜᵣᵤₙₖ×0.3=totalₜᵣᵤₙₖ

Skin segment arms:

(itchingₐᵣₘₛ+rednessₐᵣₘₛ+scalingₐᵣₘₛ+skin thicknessₐᵣₘₛ)×spreadingₐᵣₘₛ×0.2=totalₐᵣₘₛ

Skin segment head:

(itching_{head}+redness_{head}+scaling_{head}+skin thickness_{head})×spreading_{head}×0.1=total_{head}

PASI total=total_{legs}+totalₜᵣᵤₙₖ+totalₐᵣₘₛ+total_{head}

### Example 4

Participant No. 01

Initials: SG

Date of birth: Mar. 12, 1943

Sex: female

Diagnosis: psoriasis for 5 years

Dosage: neurodermatitis cocktail: 3 tablespoons per day (=20 g)

TABLE 5
PASI-Psoriasis Area and Severity Index-Patient No. 01

| | | | 2^{nd} visit after | 3^{rd} visit after |
|---|---|---|---|---|
| | | | 14 weeks | 16 weeks |
| | Baseline | 1^{st} visit after | from | from |
| PASI | visit | 6 weeks | baseline | baseline |
| Skin segment | 6.4 | 0.8 | 0.8 | 1.2 |
| legs | | | | |
| Skin segment | 0 | 0 | 0 | 0 |
| trunk | | | | |
| Skin segment | 2.4 | 0.2 | 0 | 0.2 |
| arms | | | | |
| Skin segment | 0 | 0 | 0 | 0 |
| head | | | | |
| PASI total | 8.8 | 1.0 | 0.8 | 1.4 |

Marked improvements in the PASI could be obtained for the individual skin segments as well as the PASI total. Accompanying measures comprised the patient's application of topic corticosteroids, keratolytics as well as creams and ointments free of active substances during the total supplementation period.

In the course of the study, the patient did not report any side effects of the preparation. In terms of taste, the preparation was rated “good” by the patient.

### Example 5

Participant No. 02

Initials: WA

Date of birth: May 3, 1959

Sex: female

Diagnosis: psoriasis for 3 years

Dosage: neurodermatitis cocktail: 3 tablespoons per day (=20 g)

TABLE 6
PASI-Psoriasis Area and Severity Index-Patient No. 02

| | | | 2^{nd} visit after | 3^{rd} visit after |
|---|---|---|---|---|
| | | | 9 weeks | 17 weeks |
| | Baseline | 1^{st} visit | from base- | from base- |
| PASI | visit | after 4 weeks | line | line |
| Skin segment | 2.0 | 0.4 | 0.4 | 0.4 |
| legs | | | | |
| Skin segment | 0 | 0 | 0 | 0 |
| trunk | | | | |
| Skin segment | 1.0 | 0.4 | 0.2 | 0.2 |
| arms | | | | |
| Skin segment | 0.6 | 0.2 | 0 | 0 |
| head | | | | |
| PASI total | 0.6 | 1.0 | 0.6 | 0.6 |

An improvement in the PASI could be obtained for the individual skin segments as well as the PASI total. Accompanying measures comprised the patient's application of creams and ointments free of active substances from the baseline visit till the 1st visit after 4 weeks.

In the course of the study, the patient did not report any side effects of the preparation. In terms of taste, the preparation was rated “very good” by the patient.

### Example 6

Participant No. 04

Initials: GA

Date of birth: Jun. 24, 1946

Sex: female

Diagnosis: psoriasis for 3 years

Dosage: neurodermatitis cocktail: 3 tablespoons per day (=20 g)

TABLE 7
PASI-Psoriasis Area and Severity Index-Patient No. 04

| | | | 2^{nd} visit after | 3^{rd} visit after |
|---|---|---|---|---|
| | | | 7½ weeks | 13 weeks |
| | Baseline | 1^{st} visit | from | from |
| PASI | visit | after 3 weeks | baseline | baseline |
| Skin segment | 0 | 0 | 0 | 0 |
| legs | | | | |
| Skin segment | 1.5 | 0.9 | 0.3 | 0 |
| trunk | | | | |
| Skin segment | 0 | 0 | 0 | 0 |
| arms | | | | |
| Skin segment | 0.5 | 0.2 | 0.1 | 0.4 |
| head | | | | |
| PASI total | 2.0 | 1.1 | 0.4 | 0.4 |

An improvement in the PASI could be obtained. In the trunk segment, complete remission could be achieved by the administration of the neurodermatitis cocktail. Accompanying measures comprised the patient's application of topic corticosteroids during the total supplementation period.

In the course of the study, the patient did not report any side effects of the preparation. In terms of taste, the preparation was rated “medium” by the patient.

The three reported psoriasis cases initially had PASIs of 8.8, 3.6 and 2.0, respectively. A clear and sustained improvement in the skin findings which did not change till the end of the study (PASI 1.4, 0.6 and 0.4, respectively) could be demonstrated during the neurodermatitis cocktail therapy.

The surprisingly good results of the observation study have confirmed the dietmedical therapeutic approach to using mild-temperature-concentrated mare milk as a basis for skin disease therapies. Despite the small number of cases, the success rate of 100% can be described as above-average when compared to conventional therapeutic approaches. Although the absolute content of gamma-linolenic acid was low in the nutritive mixture employed, the metabolization of alimentary precursors such as, for instance, the omega-6 fatty acid “linolic acid” could be stimulated—presumably by the activation of the enzyme delta-6-desaturase.

1-8. (canceled)

9. A method of treating neurodermatitis or psoriasis in a subject comprising:
obtaining a composition comprising a mare milk concentrate dried on a first biologically inert, disperse matrix in the absence of essential fatty acids; and
orally administering the composition to a subject, wherein neurodermatitis or psoriasis is treated in the subject.

10. method of claim 9, wherein the subject is a human.

11. method of claim 9, wherein the matrix is a highly disperse silicon dioxide.

12. The method of claim 9, wherein the composition further comprises an essential fatty acid dried on a second biologically inert, disperse matrix.

13. method of claim 9, wherein the mare milk concentrate was dried at a temperature of from 10 to 50° C.

14. The method of claim 13, wherein the mare milk concentrate was dried at a temperature of from 35 to 40° C.

15. The method of claim 9, wherein the mare milk concentrate was dried at a pressure of from 1 to 50 mbar.

16. The method of claim 15, wherein the mare milk concentrate was dried at a pressure of from 10 to 30 mbar.

17. The method of claim 9, further comprising drying the mare milk concentrate on the matrix.

18. The method of claim 12, wherein the essential fatty acid is a vegetable essential fatty acid.

19. The method of claim 9, wherein the composition further comprises at least one of hydrogen carbonate, potassium, carbonate, citrate, calcium, magnesium, vitamin C, vitamin E, niacin, zinc, iron, beta-carotene, pantothenic acid, manganese, vitamin B6, vitamin B2, vitamin B1, copper, sodium, biotin, folic acid, molybdenum, selenium, xanthan, fructose, citric acid, or vitamin B12.

20. The method of claim 9, wherein at least 330 mg of the mare milk concentrate dried on the first biologically inert, disperse matrix is administered to the subject.
